(19) United States
(12) Patent Application Publication
(10) Pub. No.: US 2010/0092390 A1
(43) Pub. Date: Apr. 15, 2010

(54) Methods for Making Particles Having Long Spin-Lattice Relaxation Times

(75) Inventors: Charles M. Marcus, Winchester, MA (US); Jacob Aptekar, Denver, CO (US); Maja Cassidy, Somerville, MA (US)

Correspondence Address: Patent Department;Choate, Hall & Stewart LLP, Two International Place, Boston, MA, 02110, US

(73) Assignee: President and Fellows of Harvard College, Cambridge, MA (US)

(21) Appl. No.: 12/248,672

(22) Filed: Oct. 9, 2008

(51) Int. Cl. A61K 49/18; B32B 1/00; B02C 17/00; B02C 23/20; B02C 23/08

(52) U.S. Cl. 424/9.3; 428/402; 241/20; 241/27

(57) ABSTRACT

Methods for making collections of small particles having spin-lattice relaxation times greater than about 5 minutes are described. The long-T₁ particles are useful as imaging agents for nuclear magnetic resonance imaging. In one embodiment, bulk silicon wafers are reduced to particles in a machining process, and the particles processed to obtain a collection of particles having an average size of about 300 nanometers and a T₁ relaxation time of about 15 minutes. The particles can be subjected to post-fabrication processing to alter their surface composition or the chemical functionality of their surface. In certain embodiments, porous particles produced by the inventive methods can be loaded with pharmaceutical drugs and used to track and evaluate delivery and effectiveness of drugs.

### GOVERNMENT FUNDING

This invention was made with United States government support under PHY-0646094 and DMR-0213805 awarded by the National Science Foundation, and 1 R21 EB007486-01A1 awarded by the National Institutes of Health. The government has certain rights in the invention.

### FIELD OF THE INVENTION

The embodiments described herein relate to methods of making particles having long nuclear magnetic, spin-lattice relaxation times. The particle sizes can be smaller than about one micron, and the spin-lattice relaxation times longer than about 5 minutes. The imaging agents are useful for various nuclear-magnetic resonance applications, and in particular magnetic resonance imaging (MRI).

### BACKGROUND

Magnetic resonance imaging (MRI) has become a powerful non-invasive diagnostic technique for viewing the internal structures of a subject or object. Currently, MRI is used routinely at medical facilities to view structures internal to patients, e.g., muscle, bone, organ structures, and to provide useful and detailed diagnostic information for attending physicians. Magnetic resonance imaging techniques are also used in the fields of geological sciences, biology and chemistry, where details about the structures of geological samples, cellular structure and function, and molecular structure can be obtained.

Details of an internal structure can be determined from a series of cross-sectional MRI images taken throughout a region of interest. Each cross-sectional image provides a two-dimensional image of the examined “slice” of the organism or material. The composite data from many cross-sectional images can provide a three-dimensional, detailed representation of the subject's or object's internal structure.

In some instances, imaging agents can be added to a subject in vivo to enhance the contrast of an MRI image. Conventional MRI contrast agents, such as those based on gadolinium compounds, operate by locally altering the spin-lattice (T₁) or spin-spin (T₂) relaxation times of the atomic nuclei. (Details about the characteristic times T₁ and T₂ are provided below.) In some cases, it is the magnetic properties of the imaging agent which can alter the local magnetic environment and affects either or both T₁ and T₂ of a native material's atomic nuclei. In some cases, imaging agents can be taken up selectively by certain types of cells or by a particular organ. The imaging agent's effect on native atomic nuclei's T₁ or T₂ values can enhance MRI contrast within the region being imaged.

Scientific research describing imaging agents having nuclei which enhance contrast in magnetic resonance imaging includes the use of ³He, ¹²⁹Xe, and ¹³C. These agents can be used for assessing lung ventilation and pulmonary and renal vascular activity. However, embodiments using these nuclei all suffer from short nuclear-magnetic-resonance enhancement periods, determined by their nuclear spin-lattice relaxation times T₁, on the order of seconds. This time is much too short to target specific cell types or track longer systemic or molecular processes.

Iron-oxide nanoparticles are also used as imaging agents in MRI techniques to monitor certain functions of biological activity. In use, the iron-oxide nanoparticles alter the local magnetic susceptibility, and thereby affect the characteristic relaxation times. Despite the ability to image these contrast agents over a one- or multiple-day uptake period, the contrast from iron-oxide suffers several limitations including difficulty quantifying the iron-oxide concentration, difficulty detecting the imaging agent in regions that undergo motion, low native signal-to-noise ratio (SNR), and an inability to distinguish the imaging agent from susceptibility artifacts and tissue background signal.

### SUMMARY

In various embodiments, the inventive methods yield nuclear-magnetic resonance, particles having long spin-lattice relaxation times, T₁. The particles can be biocompatible and manufacturable at low cost. In various embodiments, the particles can be used as an imaging agent for nuclear magnetic resonance (NMR) applications. In certain embodiments, the spin-lattice relaxation times T₁ for the particles are longer than about 5 minutes, longer than about 15 minutes, longer than about 30 minutes, longer than about one hour, longer than about two hours, and longer than about three hours. These particles can provide enhanced signal-to-noise quality in certain nuclear-magnetic resonance applications, e.g., magnetic resonance imaging (MRI). In various embodiments, the inventive methods can be used to produce a collection of micro- or nanoparticles having a selected particle size distribution and a characteristic spin-lattice relaxation time T₁ greater than about 15 minutes. The particle size distribution can be determined by multiple steps of centrifugation. Particle sizes can be any value between about 10 nanometers and about 200 microns.

In various embodiments, methods for making particles having long T₁ times include obtaining a substantially pure material comprising at least one chemical constituent present within the material in at least one form having nuclear spin not equal to zero and a spin-lattice relaxation time T₁ greater than about 5 minutes. The methods further include steps of reducing the substantially pure material into particles in the presence of one or more solvents, and separating the particles by size to yield one or more collections of particles exhibiting a spin-lattice relaxation time greater than about 5 minutes. In some embodiments, a yielded collection of particles comprises nanoparticles with substantially the same crystal structure and doping characteristics as the substantially pure material.

In some embodiments, a yielded collection of particles comprises a collection of micro- or nanoparticles having a selected particle size distribution and a characteristic spin-lattice relaxation time T₁ greater than about 15 minutes. In some embodiments, a yielded collection of particles has an average particle size between about 1 nanometer and about 200 microns and a characteristic spin-lattice relaxation time T₁ greater than about 15 minutes. In certain embodiments, a yielded collection of particles can further be characterized by a particle size distribution, wherein more than about 90% of the particles have a size within a range between about plus 60% and about minus 60% (+60%) of the average particle size. In some embodiments, the particle size distribution is such that more than about 90% of the particles have a size within a range between about ±40% of the average particle size.

The inventive aspects include methods where the long-T₁ particles are delivered internally to one or more cells, an organism, a specimen, a system, or a living subject, and used as an imaging agent in MRI applications. The method of delivery can include the steps of receiving particles having spin-lattice relaxation times T₁ greater than about 5 minutes, and delivering a selected quantity of the particles internally to one or more cells, an organism, a specimen, a system, or a living subject.

Various inventive methods include fabricating surface-modified particles for nuclear-magnetic resonance applications, e.g., magnetic resonance imaging. In some embodiments, the method of fabricating surface-modified particles includes the steps of receiving particles having spin-lattice relaxation times T₁ greater than about 5 minutes, and coating the particle with a passivating and/or biologically compatible moiety wherein the passivating moiety provides a protective layer enabling the particle to withstand a living system's natural defense against foreign bodies. In certain embodiments, a method for surface modification can include the steps of receiving particles having spin-lattice relaxation times T₁ greater than about 5 minutes, and chemically functionalizing the surface of the particles so that the particle binds specifically to a desired target cell type, molecule, or molecular expression.

In some inventive methods, the particles may be loaded with a pharmaceutical drug prior to delivery to a subject or specimen. A method for fabricating drug-carrying particles for magnetic resonance imaging can comprise receiving porous particles having spin-lattice relaxation times T₁ greater than about 5 minutes, and subjecting the porous particles to a drug-loading process, wherein the particles are exposed to a drug to be loaded into the vacancies of the particles. The pharmaceutically-loaded particles can be administered to a subject or specimen and used to track drug delivery within the subject or specimen.

The foregoing and other aspects, embodiments, and features of the present teachings can be more fully understood from the following description in conjunction with the accompanying drawings.

### BRIEF DESCRIPTION OF THE DRAWINGS

The skilled artisan will understand that the figures, described herein, are for illustration purposes only. In the drawings, like reference characters generally refer to like features, functionally similar and/or structurally similar elements throughout the various figures. The drawings are not necessarily to scale, emphasis instead being placed upon illustrating the principles of the teachings. The drawings are not intended to limit the scope of the present teachings in any way.

FIG. 1 represents the dynamics of motion of a nuclear magnetic moment 110 in a substantially uniform and static magnetic field \vec{B}. The magnetic moment will precess, tracing out path 120 in gyroscopic motion.

FIG. 2A represents a collection of atoms 210 for which the magnetic moments 110 are randomly oriented.

FIG. 2B represents a collection of atoms that have been polarized by a magnetic field \vec{B}. A fraction of the atoms 220 have their magnetic moments oriented in a direction substantially aligned with the magnetic field.

FIGS. 3A-3C illustrates a hyperpolarized collection of atoms comprising a particle for which the nuclear spin is weakly coupled to the atom's electron cloud. As the particle tumbles, the magnetic moments substantially maintain their orientation in space, irrespective of the particle's orientation.

FIG. 4 depicts a method for making particles having long spin-lattice nuclear-magnetic relaxation times T₁.

FIG. 5 depicts an embodiment of a ball-mill machining process 500 for making small particles having long T₁ relaxation times. A substantially pure material 520 is placed in a ball mill drum 550 along with a solvent or liquid 530 and milling balls 510. The drum is covered and rotated at a selected rotation speed for a selected amount of time. The machining process reduces the material 520 into a collection of small particles, e.g., a powder. The powder is subjected to subsequent processing steps to yield particles having long T₁ relaxation times.

FIGS. 6A-6C depict various embodiments of steps employed in making NMR-active particles having long-T₁ characteristics.

FIG. 7A depicts various embodiments of post-fabrication methods for particles having long spin-lattice relaxation times.

FIG. 7B depicts embodiments of methods for administering imaging agent particles having long spin-lattice relaxation times.

FIG. 8A is a plot of particle size distribution for a solution containing particles formed by the inventive methods.

FIG. 8B is a plot of saturation recovery for the particles in solution reported in FIG. 8A.

FIG. 8C is a plot of the average of two measurements of the NMR spectra for the particles reported in FIG. 8A.

FIG. 9A reports size distributions of small particles remaining in a supernatant after light centrifuging at about 3,500 RCF.

FIG. 9B reports various size distributions obtained after separating the particles by size. The supernatant of FIG. 9A was subjected to additional steps of centrifuging to yield the various size distributions.

FIG. 10 is a plot of experimental data showing T₁ times measured for various collections of particles, each characterized by an average particle size. Data is shown for two types of silicon, high resistivity and low resistivity, used to produce the particles.

The features and advantages of the present invention will become more apparent from the detailed description set forth below when taken in conjunction with the drawings.

### DETAILED DESCRIPTION

### I. Introduction

The inventive methods described herein are useful for manufacturing small particles having long spin-lattice relaxation times (long T₁ times), e.g., longer than about 5 minutes. These particles can be used for various applications in the field of nuclear magnetic resonance, e.g., magnetic resonance imaging (MRI). In various embodiments, a form of bulk material, e.g., amorphous, crystalline, porous, polycrystalline or nanocrystalline, having a suitable composition of a long-T₁ constituent can be reduced to micron-scale, sub-micron-scale and/or nanometer-scale particles, e.g., particles ranging in size from about 1 micron to about 200 microns, from about 200 nanometers to about 1 micron, and from about 1 nanometer to about 200 nanometers, and retain the long-T₁ characteristics. The bulk material can be reduced to particles by certain machining and processing steps. Collection of particles having different size distributions can be produced by centrifugation or filtration of the machined particles, or a combination of both centrifugation and filtration techniques. Additional steps may be carried out which reduce the presence of contaminants on the particles and alter the surface properties of or provide chemical functionality to the small particles. In some embodiments, the particles are manufactured at low cost.

Applications for MRI imaging agents having long T₁ times include medical diagnosis and evaluation of systemic, cellular and molecular biological functions. The particles can be used as imaging agents for MRI applications including, but not limited to, neurological disorders, cancer diagnosis and staging, and diseases of the lungs, brain, heart, intestines, pancreas, liver and kidneys. Angiography, perfusion, cell tracking, and receptor-ligand targeting are additional potential applications for the long-T₁ imaging agent. In some embodiments, the particles are coated or treated prior to use, e.g., have their surfaces chemically modified or functionalized. In some embodiments, the particles are uncoated or untreated prior to their use. The imaging agents can be used to identify the presence of a disease, track the delivery of drugs, and monitor the progressive/regressive response to therapies. In some embodiments, the imaging agents can be used as tags in high-throughput in vitro assays to detect whether certain ligands or drugs reach their intended targets. In some embodiments, the particles can be delivered or infused into non-living samples, e.g., geological specimens, for MRI analysis.

### II. Definitions

The following definitions are set forth to illustrate and define the meaning of various terms used to describe the embodiments herein.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Imaging agent particles: As used herein, the term “imaging agent particles” refers to particles having nuclear magnetic resonance properties. In certain embodiments, the spin-lattice relaxation time T₁ of imaging agent particles is greater than about 5 minutes.

Micron-scale: As used herein, the term “micron-scale” refers to particles having a maximum diameter or dimension in a range between about 1 micron and about 200 microns. (1 micron=10⁻⁶ meter)

Nanometer-scale: As used herein, the term “nanometer-scale” refers to particles having a maximum diameter or dimension in a range between about 1 nanometer and about 200 nanometers. (1 nanometer=10⁻⁹ meter)

Particles: As used herein, the term “particles” generally refers to micron-scale, submicron-scale, and/or nanometer-scale particles

Substantially: As used herein, the term “substantially” refers to the qualitative condition of exhibiting total or near-total extent or degree of a characteristic or property specified.

Submicron-scale: As used herein, the term “submicron-scale” refers to particles having a maximum diameter or dimension in a range between about 200 nanometers and about 1 micron.

### III. Aspects of Magnetic Resonance Imaging

By way of introduction to the inventive methods, several aspects of magnetic resonance imaging, nuclear magnetic resonance, spin-lattice relaxation times T₁, and spin-spin relaxation times T₂ are reviewed briefly.

In overview, magnetic resonance imaging (MRI) relies on nuclear magnetic resonance (NMR) properties of atoms, and how these properties are affected by their local environment. Generally, when an atom having a nuclear magnetic moment, i.e., a non-zero nuclear spin, is placed in a magnetic field, the magnetic moment precesses in gyroscopic motion about an axis substantially aligned with the external magnetic field. The precessing moments for a selected species of atoms can be probed by applying radio-frequency (RF) electromagnetic fields tuned to the species' precessional resonance frequency ω, and resulting signals can be detected to provide data useful for constructing spatial images of the distribution of the selected species of atoms. The strength of the resulting signals, the resonance frequency (o, and the signals' rates of decay depend upon several factors including the type of atom being probed and its local environment.

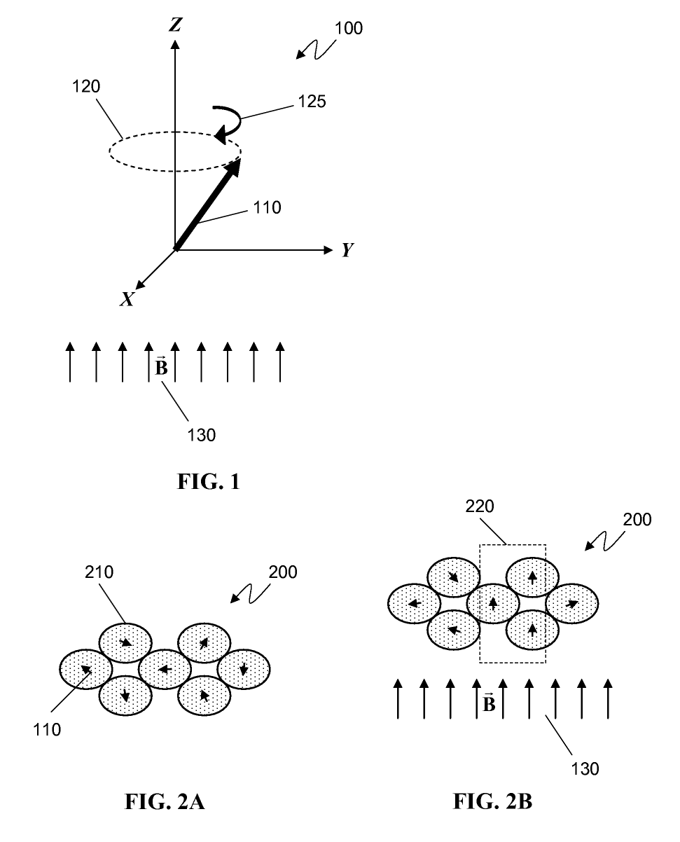

Referring now to FIG. 1, the diagram depicts the dynamics of motion 100 for an atom's nuclear magnetic moment 110 placed in a magnetic field 130. A single nuclear magnetic moment 110 will precess about an axis, e.g., the Z axis, which is substantially collinear with an applied static magnetic field \vec{B} 130 as indicated in the figure. The precessional frequency ω depends in part upon the strength of the local magnetic field, e.g., the field in the immediate vicinity of the atom. In the illustration, the magnetic moment 110 precesses about the Z axis, tracing out the path 120 in the direction indicated by arrow 125.

FIG. 2A represents a collection of atoms or molecules. The ensemble 200 may contain many individual atoms or molecules 210, each with a nuclear magnetic moment 110. In some embodiments, not every atom or molecule 210 may have a non-zero nuclear magnetic moment 110. In some embodiments, a minority of the atoms or molecules within the ensemble may have non-zero magnetic moments. In certain embodiments, the ensemble 200 constitutes a nanoparticle.

The magnetic moments 110 of an ensemble 200 placed in a substantially uniform and static magnetic field 130 will tend to reorient along the direction of the applied field. This reorientation is referred to as a polarization of the magnetic moments. FIG. 2B illustrates polarization of an ensemble of atoms. The magnetic moments 110 of a fraction of the atoms 220 have reoriented in a direction substantially aligned with the applied static magnetic field 130, and the particle 200 takes on a net magnetic moment. When the applied external magnetic field is removed, the orientation of the atoms' moments will randomize at a characteristic rate referred to as the longitudinal relaxation time or the “spin-lattice” relaxation time, T₁. Referring to FIG. 1, during randomization the direction of the magnetic moment 110 for any atom may drift in time, away from the path 120, and may point in the −Z direction at a later time. The randomization of all magnetic moments within the ensemble can result in zero net magnetic moment for the particle, as depicted in FIG. 2A.

In some embodiments, MRI images of a subject or specimen are obtained by measuring a signal associated with the spin-lattice, T₁, relaxation time for finite regions, called voxels, within the subject. In various embodiments, a region to be imaged is divided into multiple smaller voxels. Variations in the local density and material composition may alter the T₁ time and associated signal from voxel to voxel. In certain medical imaging embodiments, the relaxation time of the hydrogen nucleus (H⁺) may be measured. Changes in the relaxation time from voxel to voxel, due to variations in the local environment, yields information about the internal structure of the subject.

When nuclear magnetic moments for a collection of atoms are polarized and maintained in a substantially static magnetic field, their precessional motion can be substantially synchronized by the application of an RF electromagnetic field tuned to match approximately the precessional frequency ω. The applied field tends to force the precessing moments 110 into synchronous motion. When the RF field is removed, the precessing moments begin to drift out of phase with one another. This rate of de-phasing of precessional motion is referred to as the transverse relaxation time or the “spin-spin” relaxation time, T₂. Referring again to FIG. 1, a collection of atoms having their magnetic moments 110 synchronized would exhibit precessional motion 125, 120 in phase with each other.

Often, MRI signals are derived from the transverse relaxation properties of the sample. In such techniques, sequences of RF fields may be applied to the sample. In one embodiment, a short-duration RF field may be applied to synchronize the moments' precessions. After a brief delay, another short-duration RF field may be applied to flip the spin orientation of the nuclear moments. This would correspond to changing the moment's 110 orientation from the +Z direction to the −Z direction in FIG. 1. The spin reversal causes the formerly de-phasing moments to drift back into phase producing a large detectable magnetic impulse when resynchronized. This measurement technique can be repeated many times at a rate on the order of about twice the transverse relaxation time, T₂, to improve the signal-to-noise ratio when collecting data for producing images.

Some difficulties can arise in MRI when the local environment unfavorably affects the spin-lattice relaxation time T₁ or the spin-spin T₂ relaxation time. Regarding T₁, the local environment may rapidly randomize the orientation of the atoms' magnetic moments so that T₁ is very short, for example less than milliseconds or microseconds. When the orientation of the magnetic moments are randomized, nuclear magnetic resonance signals derived from T₂ measurements can no longer be obtained from the sample. A short T₁ can result in a degradation of the signal-to-noise ratio and imaging resolution.

Regarding T₂, two functionally dissimilar species may be in an environment where they are nearly physically indistinguishable, in terms of their MRI characteristics. For example, scarred muscle tissue may be semi- or non-functional but be physically indistinguishable, in terms of its MRI signature, from surrounding healthy muscle tissue. The two types of muscle tissue may have substantially the same transverse relaxation times T₂. As another example, cancerous growth within an organ may initially go undetected because of its similar MRI characteristics to the surrounding cells from which it has replicated. For such cases, even though the signal-to-noise ratio may be adequate, the contrast of the examined species may be so low as to go undetected by MRI.

### IV. Methods for Making Small Particles with Long-T₁ Times

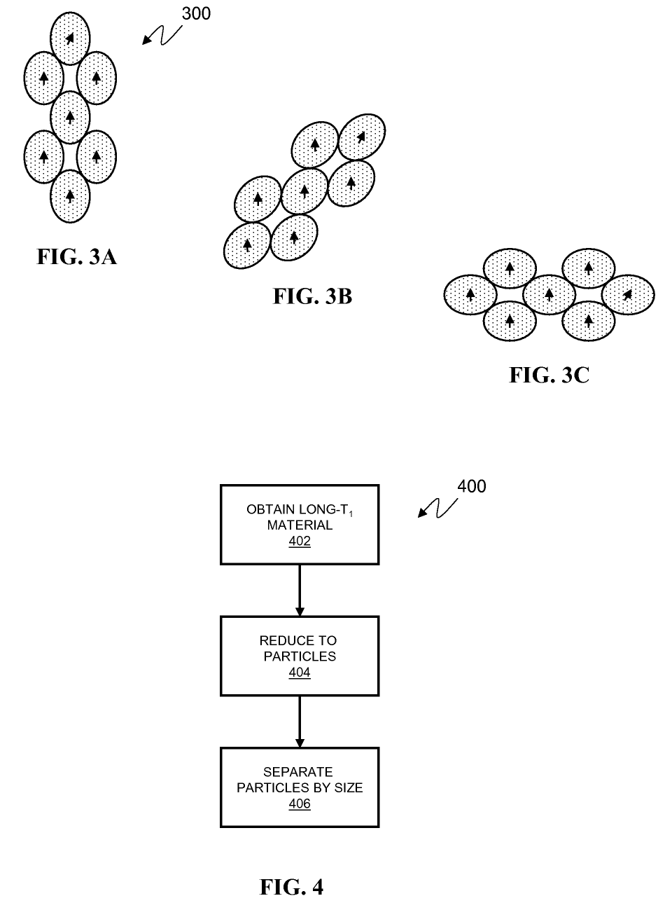

The inventors have devised methods for making small particles which exhibit long-T₁ times. In certain embodiments, the particles can be hyperpolarized and used as an imaging agent for MRI applications. They can have a substantial fraction of their non-zero nuclear magnetic moments polarized along a preferred direction as indicated in FIG. 3A, and maintain their polarization for long periods while being delivered to a target site within one or more cells, an organism, a specimen, a system, or a living subject. In various embodiments, the imaging agents provide NMR signals long after delivery of the imaging agent. In this context, long periods associated with T₁ relaxation times or long-T₁ times refers to periods longer than about 5 minutes in some embodiments. In various embodiments, the T₁ time is longer than about 15 minutes, longer than about 30 minutes, longer than about one hour, longer than about two hours, and yet in some embodiments longer than about three hours.

In various embodiments, the inventive particles maintain their long-T₁ properties when formed in small sizes by the inventive methods. The particles can be formed into micron-sized, sub-micron-sized, and nanometer-sized particles. In certain embodiments the particles can be hyperpolarized prior to delivery into one or more cells, an organism, a specimen, a system, an in vitro assay, or a living subject. Delivery can occur by various methods, e.g., injection, infusion, ingestion, implantation, absorption and inhalation. Magnetic resonance images can be acquired by detecting signals from the particles over long time durations. In living systems, the images can represent the spatial and temporal biodistribution of the particles, and can be used as a functional augmentation to conventional anatomical proton (H⁺) MRI. In some embodiments, images obtained using long-T₁ particles may be overlayed with images obtained using conventional anatomical proton (H⁺) MRI.

The inventors have recognized that bulk silicon (Si) can exhibit long spin-lattice relaxation times T₁, and is receptive to hyperpolarization. Additionally, silicon is biocompatible and biodegradable, and not normally present in high abundance in living subjects. Because of its low abundance in living subjects, the inventors postulated that small silicon particles may provide improved signal-to-noise quality in certain NMR applications. Additional materials proposed that may potentially exhibit long-T₁ relaxation times include, but are not limited to, compound forms of silicon, e.g., silicon dioxide, silicon nitride, and silicon carbide, carbon, and compound forms of carbon. In various embodiments, imaging agents formed into micron-sized or submicron-sized particles from silicon, a silicon compound, carbon, or a carbon compound can exhibit long-T₁ relaxation times, provided the process of forming the particles does not significantly and adversely affect the nuclear magnetic resonance properties of the material.

The inventors have also recognized that silicon and carbon may be formed into a particular crystal structure which exhibits a weak coupling between the atoms' electrons and nuclear spin. When formed in the diamond crystal structure, the electron environment, from the perspective of an atom's nucleus, is substantially isotropic. The resulting weak coupling between the electrons and nuclear spin substantially decouples the nuclear magnetic moments' orientation from the crystal lattice. That is, the orientation of the nuclei's moments are not locked to the orientation of the material. This effect is illustrated in FIGS. 3A-3C. A hyperpolarized particle 300 initially has its magnetic moments substantially aligned vertically. As the particle moves and tumbles, the nuclear magnetic moments can remain substantially aligned in the vertical direction. This decoupling is desirable for magnetic resonance imaging agents, where the particles may be hyperpolarized prior to being administered to a subject or specimen.

### IV-A. Methods for Making the Particles

Referring now to FIG. 4, a flow chart 400 depicts an embodiment of a method of making micron-sized, submicron-sized, or nanometer-sized imaging agents having long-T₁ relaxation times. In various embodiments, the method comprises a step of obtaining 402 a material having one or more atomic species therein exhibiting long spin-lattice relaxation times. In some embodiments, the constituent atomic species' spin-lattice relaxation time is longer than about 5 minutes. In certain embodiments, the T₁ time is longer than about 15 minutes, longer than about 30 minutes, longer than about one hour, longer than about two hours, and yet longer than about three hours. The method may further comprise a step of reducing 404 the material to particles. The particles may have a range of sizes, at least some being between about 10 nanometers and about 200 microns in size. The method may further comprise a step of separating 406 the particles by size, e.g., separating out one or more powders or collections of particles wherein the particle size range within each collection differs from the size range within other powders or collections. As an example, one powder may contain particles with sizes between about 10 nanometers and about 100 nanometers, another may contain particles with sizes between about 80 nanometers and about 300 nanometers, and another powder may contain particle sizes between about 400 nanometers and about 600 nanometers. In some embodiments, the separated size ranges may be overlapping. In some embodiments, the separated size ranges may be non-overlapping.

### IV-A-1. Obtaining Material

In various embodiments, methods for making small particles for magnetic resonance imaging include obtaining 402 a substantially pure material comprising at least one chemical constituent having spin-lattice relaxation times greater than about 5 minutes present within the material. In certain embodiments, the constituent may be present as an isotope which has non-zero nuclear spin. In some embodiments, the substantially pure material can be selected from the following group of materials: silicon, silica, silicon carbide, silicon nitride, carbon, diamond and nano-diamond. The stoichiometric purity of the material may be greater than about 90%, greater than about 95%, greater than about 99% in some embodiments, greater than about 99.9% greater than about 99.99%, greater than about 99.999%, and greater than about 99.9999% in some embodiments. The material's form may be any of the following types: amorphous, crystalline, porous, polycrystalline, co-crystalline or nanocrystalline.

In various embodiments, the material may be in a form for which the nuclear spin is substantially decoupled from the electron cloud for certain constituent atomic species having non-zero nuclear spin. For example, the silicon isotope ²⁹Si has a natural abundance of about 4.7% in bulk silicon, and is a spin-one-half nucleus that can be detected with magnetic resonance techniques. Silicon can be formed in bulk having a diamond lattice structure. In certain embodiments, the bulk silicon may comprise substantially three silicon isotopes: ²⁸Si (zero nuclear spin, about 92.2% abundant), ²⁹Si (spin=½, about 4.7% abundant) and ³⁰Si (zero spin, about 3.1% abundant). Any form of bulk silicon—amorphous, crystalline, polycrystalline, porous, nanocrystalline, or cocrystalline,—of adequate purity may be used for the inventive methods of making particles having long T₁ times. Bulk crystalline silicon of high purity, greater than about 99.9999%, has a resistivity ranging from about 1 kiloOhm-cm to about 100 kiloOhm-cm, has T₁ relaxation times near five hours, and is available from Silicon Quest International, Inc. of Santa Clara, Calif. In some embodiments, bulk silicon having a purity greater than about 99.9999% and having a resistivity between about 1 kiloOhm-cm (kΩ-cm) and about 100 kiloOhm-cm is used to make particles having long T₁ times according to the inventive methods described herein. As another example, the carbon isotope ¹³C has a natural abundance of about 1.1% in bulk carbon, and also is a spin-one-half nucleus that can be detected with magnetic resonance techniques. Carbon can also be formed into a diamond structure.

In some embodiments, bulk silicon of a selected purity and a selected resistivity is used to make the small particles. The selected resistivity can be between about 10 kΩ-cm and about 100 kΩ-cm, between about 30 kΩ-cm and about 100 kΩ-cm, between about 50 kΩ-cm and about 100 kΩ-cm, and yet in some embodiments between about 75 kΩ-cm and about 100 kΩ-cm. In some embodiments, the resistivity of the bulk silicon is higher, e.g., up to 150 kΩ-cm, or up to 200 kΩ-cm.

In various embodiments, the abundance of the non-zero nuclear spin isotope in the obtained material may differ from its natural abundance. In some embodiments, the abundance of an isotope may be altered. In certain embodiments, the concentration of the element present in the form having nuclear spin not equal to zero may be any value between about 0.1% and about 100% of the total material present. For example in reference to silicon, the concentration of ²⁹Si may be higher than its natural abundance, e.g., higher than about 4.7%, higher than about 5%, higher than about 7%, higher than about 10%, higher than about 20%, higher than about 30%, higher than about 40% or even higher than about 50%. In yet another embodiment, the level of ²⁹Si may be lower than its natural abundance level, e.g., lower than about 4.7%, lower than about 4%, lower than about 3%, lower than about 2%, lower than about 1%, lower than about 0.5% or even lower than about 0.1%. Methods for preparing silicon materials, e.g. silicon (Si) or silica (SiO₂), with varying levels of silicon isotopes have been developed for the computer industry, e.g., see Haller, J. Applied Physics 77:2875, 1995.

In some embodiments, dopants may be intentionally incorporated in the substantially pure material. The dopants may alter the spin-lattice relaxation time of the non-zero spin constituents, and may be incorporated after obtaining the material, e.g., by ion implantation, or may have been incorporated prior to obtaining the material, e.g., n-type or p-type dopants may have been added to silicon during crystal growth. The T₁ times of ²⁹Si in silicon doped with various levels of n-type or p-type dopants have been investigated in Shulman and Wyluda, Phys. Rev. 103:1127, 1956. The T₁ times of ²⁹Si ranged from hours to minutes when the mobile carrier concentration was adjusted from about 1×10¹⁴ cm⁻³ to about 1×10¹⁹ cm³ with the incorporation of the dopants. The n-type dopants had a greater impact on T₁ times. It will be appreciated that any of a variety of dopant types or doping levels can be used to alter T₁ times.

In certain embodiments, a trade-off may exist between longer T₁ times and ease of hyperpolarization of the material. For example, a material which exhibits a long T₁ time may require higher magnetic fields and/or longer immersion times within the magnetic field to hyperpolarize the material than are required for materials which exhibit shorter T₁ times. The appropriate combination of T₁ time and ease of hyperpolarization may determine the selection of material for a particular application. Some applications may favor very long T₁ times, and thus require lower dopant levels. Other applications may not require long T₁ times, and may therefore tolerate higher dopant levels. Accordingly, in various embodiments, a material may be selected for a particular application based upon its T₁ time and/or its dopant level. In some embodiments, a material may be selected according to a particular concentration of dopant, e.g., bulk silicon with one of various dopant concentrations available commercially from Virginia Semiconductor of Fredericksburg, Va. In some embodiments, a particular dopant concentration can be achieved using methods known in the semiconductor art and disclosed in Haller, J. Applied Physics 77:2857, 1995.

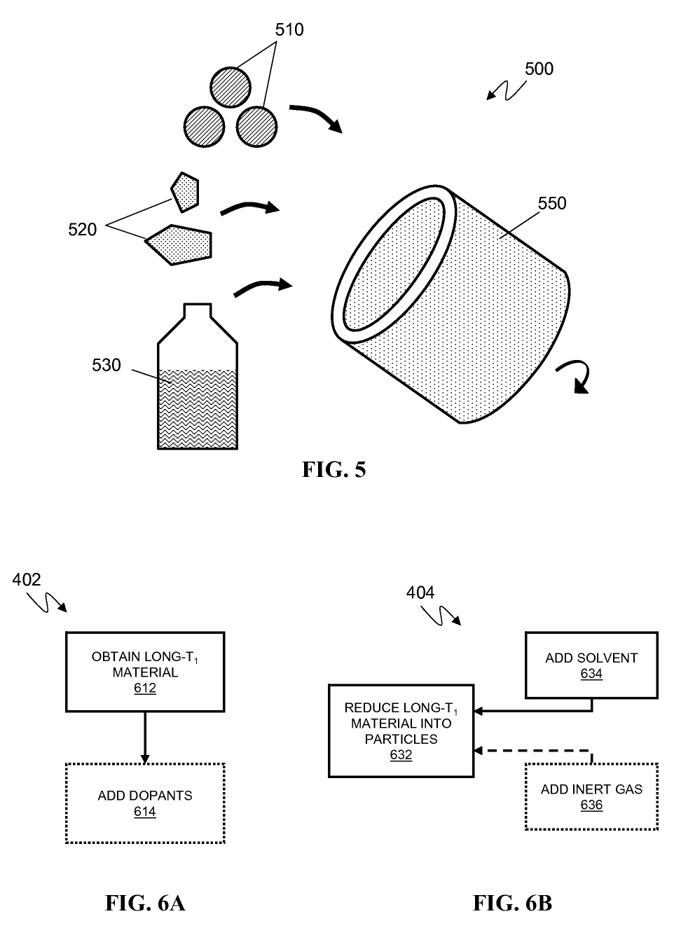

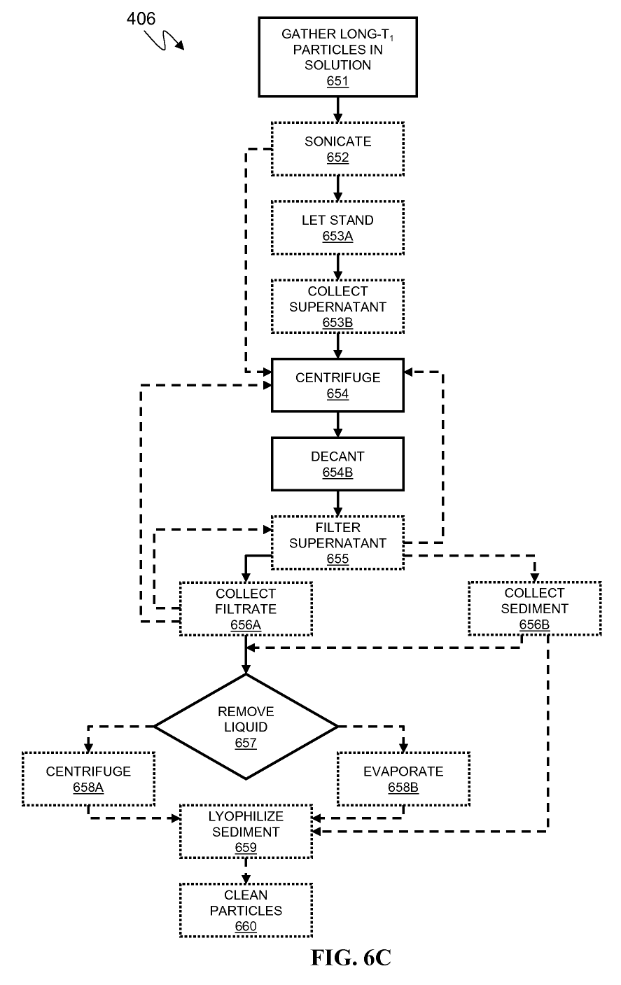

For purposes of this application, the incorporation of dopants into the material does not constitute increasing the level of impurities in the material. Impurities are defined herein as elements, compounds, particles, or defects which are not intentionally introduced into the substantially pure material. In various embodiments, the stoichiometric purity of the material, used to form particles, may be greater than about 90%, greater than about 95%, greater than about 99%, greater than about 99.9%, greater than about 99.99%, greater than about 99.999%, greater than about 99.9999%. The concentration of the element present in the form having nuclear spin not equal to zero may be any value between about 0.1% and about 100% of the total material present. FIG. 6A depicts an embodiment of a method for obtaining material 402 which provides for the addition of dopants to the material. For example, dopants may be added by ion implantation. The step of adding dopants 614 is optional and is indicated as a dotted box.

IV-A-2. Reducing the Material into Particles

Once a suitable material is obtained, a method for making small particles having long T₁ relaxation times may further include the step of reducing 404 the substantially pure material into particles in the presence of one or more solvents. As an example of a step of reducing 404 the material to particles, the material may be subjected to a machining process 500 as depicted in FIG. 5. The illustration depicts a ball milling process, in which substantially pure material 520 having desirable nuclear magnetic resonance properties, is placed in the drum 550 of a ball mill. Milling balls 510 are placed in the drum 550, and a solvent or liquid 530 is added. The balls 510 may be alumina milling balls about 10 millimeters in diameter. In some embodiments, the balls 510 may be zirconia milling balls. Balls of other diameters may be used in some embodiments, e.g., balls having diameters of between about 2 mm and about 15 mm, between about 15 mm and about 25 mm, and yet between about 25 mm and about 50 mm. In some embodiments, two or more sets of milling balls may be used. For example, a first set having a particular diameter between about 15 mm and about 25 mm may be used for a first period of reducing the bulk material 520 into particles, and a second set having a particular diameter between about 2 mm and about 15 mm may be used for a second period of reducing the bulk material into particles. In some embodiments, isopropanol is used as the milling solvent or liquid 530 and added into the drum to reduce particle agglomeration. In some embodiments, ethanol is used as the milling solvent or liquid 530 and added into the drum to reduce particle agglomeration. The drum 550 may then be covered and rotated at a selected speed for a selected amount of time. In various embodiments, the rotation speed can be any value in a range between about 0 revolutions per minute (RPM) and about 50 RPM, between about 50 RPM and about 500 RPM, and between about 500 RPM and about 1,000 RPM. In certain embodiments, the selected amount of time for the machining is between about 1 minute and about 12 hours, between about 12 hours and about 48 hours, and yet between about 48 hours and about 96 hours. The action of rotation and tumbling of the balls 510 and material 520 reduces the bulk material into a powder or collection of particles of various sizes. At least some of the particles may range in size from about a few nanometers to about tens of microns.

In certain embodiments, the ball mill may be operated at several different speeds during the milling process. For example, the ball mill may be initially operated at a low speed, e.g., between about 50 RPM and about 100 RPM, for a first period of time after the material 520 and solvent or liquid 530 are added to the drum 550. The mill may then be operated at a higher speed, e.g., between about 100 RPM and about 500 RPM, for a second period of time. In some embodiments, the reducing of the material into particles may be carried out intermittently. For example, a ball mill may be operated at a first speed for a first period of time, and then left to stand idle for a second period of time. The mill may then be operated at a second speed for a third period of time.

In some embodiments, the reducing 404 of the bulk material to particles can be carried out in an inert gas environment, e.g., an argon, helium or nitrogen environment. In certain embodiments, an inert gas environment can prevent the occurrence of unwanted reactions on the surface of the particles. For example, machining in a pure nitrogen environment may reduce oxidation on the surface of the silicon particles.

Various types of instruments may be used for reducing 404 the bulk material to particles, and various solvents or liquids 530 may be used during the reducing step. In various embodiments, the step of reducing 404 may comprise reducing 632 the long-T₁ material into particles. In some embodiments, a jet mill, a grinding machine, a drilling machine, a cutting machine, a ball mill, or a combination thereof may be used to reduce the bulk material into particles. In some embodiments the solvent used during the step of reducing 632 may be purified water, de-ionized water, distilled water, ethanol, isopropanol, methanol, acetone, an oil or cutting fluid, or a combination thereof. FIG. 6B depicts an embodiment of a method for reducing the long-T₁ material into particles 630, and provides steps of adding a solvent or liquid 634 and optionally adding an inert gas 636.

### IV-A-3. Separating Particles by Size

After the step of reducing 404, the particles of the substantially pure material can be gathered in solution, and subjected to a step of separating 406 by size to yield one or more powders or collection of particles of the substantially pure material. In various embodiments, the sizes of particles within a yielded powder can be any values between about 1 nanometers (nm) and about 200 nm, e.g., nanometer-scale particles, between about 200 nm and about 1 micron, e.g., sub-micron-scale particles, between about 1 micron and about 200 microns, e.g., micron-scale particles. In certain embodiments, sizes of the produced particles may straddle one or more of these size ranges, e.g., particles may be yielded with a size range between about 80 nm and about 300 nm, between about 400 nm and about 2 microns, etc.

In various embodiments, the size range of produced particles is determined by the separation techniques employed. For example, in certain methods, particular centrifuge speeds and/or filter pore sizes are selected to produce a collection of particles having a particular size range. In various embodiments, separation techniques are designed and selected to produce one or more desired size ranges.

In various embodiments, particles within the powders have spin-lattice relaxation times T₁ greater than about 5 minutes, greater than about 15 minutes, greater than about 30 minutes, greater than about one hour, greater than about two hours, and greater than about three hours. The process of separating 406 the particles by size may be carried out in several steps, and may further include optional steps, as depicted in FIG. 6C. Various embodiments of the step of separating 406 by size the particles having long T₁ relaxation times are depicted in FIG. 6C. Dotted boxes indicate optional steps, and dashed lines indicate optional flow paths. The step of separating 406 may further comprise a cleaning step 660.

In various embodiments, the long-T₁ particles are gathered in solution 651 after the step of reducing 632. The solvent or liquid can be ethanol. Particles can then be sonicated 652 for a period of time less than about 10 minutes, and left to stand idle 653A for a period of about 48 hours, so that larger particles collect as sediment at the bottom of the vessel. In some embodiments, the sonication 652 can be for less than 5 minutes, less than 2 minutes, and less than 1 minute. In various embodiments, the amount of time the solution is left to stand idle 653A can be a period of time between about 1 hour and about 12 hours, between about 12 hours and about 48 hours, between about 2 days and about 4 days, and yet between about 4 days and about 8 days.

In some embodiments, after the sonicated solution stands idle for a period of time, at least a portion of the solution is centrifuged 654. In some embodiments, after the sonicated solution stands idle for a period of time, at least a portion of the sonicated solution's supernatant is collected 653B and centrifuged 654, and subjected to further processing steps. In various embodiments, the light centrifuging 654 removes particles of sizes greater than or less than a selected size. The selected size can be determined by centrifugation speed, duration of centrifugation, and/or filling height of the centrifuge tube. In certain embodiments, the centrifuging removes particles of sizes greater than about 10 microns, greater than about 5 microns, greater than about 1 micron, greater than about 700 nm, greater than about 400 nm, greater than about 300 nm, greater than about 200 nm, and yet greater than about 100 nanometers from the centrifuged solution when the supernatant is taken for use or further processing. In certain embodiments, the light centrifuging removes particles of sizes less than 5 microns, less than 1 micron, less than about 700 nm, less than about 400 nm, less than about 300 nm, less than about 200 nm, and yet less than about 100 nm from the centrifuged solution when the pellet is taken for use or further processing.

In some embodiments, the centrifuge may be operated at about 3,500 relative centrifugal force (RCF) for about 5 minutes. In various embodiments, the centrifuging 654 may be carried out between about 1,500 RCF and about 2,500 RCF, between about 2,500 RCF and about 4,500 RCF, and in some embodiments between about 4,500 RCF and about 7,000 RCF, for periods of time between about 1 minute to about 2 minutes, about 2 minutes to about 4 minutes, about 4 minutes to about 8 minutes, and about 8 minutes to about 16 minutes.

In some embodiments, the sonicated solution can be centrifuged directly after sonication 652. In this embodiment, steps of letting the solution stand idle 653A and collecting the supernatant 653B may be omitted. The centrifugation can precipitate large particles out of solution, which would otherwise have settled out during the step of letting the solution stand idle 653A.

Before or after the step of centrifuging 654, or both before and after, the supernatant can be filtered 655. The filtering can be carried out in one or more steps using filters with graduated pore sizes to yield a solution having particles suspended therein of a maximum size. For example, the filtering 655 can begin with a filter having a pore size of about 10 microns. The filtrate may then be collected 656A and filtered in a subsequent filtering step 655 with a filter having a smaller pore size. For example, subsequent steps may use filters having gradually reducing pore sizes, e.g., about 5 microns, about 2 microns, about 1 micron, about 500 nanometers, about 200 nanometers, about 100 nanometers, etc. Any combination of the aforementioned filters, or subsets thereof, may be used, including filters having substantially identical pore sizes. In some embodiments, the filtering 655 may be carried out in separate sequential steps. In some embodiments, the filtering 655 may be carried out in a single step with a filtering apparatus that incorporates a sequential set of filters having gradually reducing pore sizes.

In various embodiments, the filtered sediment from any filtering step 655 may be collected 656B for further processing. For example, after an individual filtering step with a filter having a pore size of about 200 nm, following a previous step with a filter having a pore size of about 500 nm, the filtered sediment may be collected 656B from the filter having 200 nm pore sizes to yield particles with a size range between about 200 nm and about 500 nm.

After completing one or more filtering sequences, liquid is removed 657 from the filtrate. The filtrate can be centrifuged vigorously 658A to precipitate the suspended particles out of the solution. The vigorous centrifuging 658A can be carried out at about 12,000 RCF for about 10 minutes. In various embodiments, the centrifuging can be carried out at an RCF ranging between about 7,500 and about 10,000, between about 10,000 and about 15,000, and between about 15,000 and about 20,000. The centrifuging 658A may be carried out for time periods between about 2 minutes to about 4 minutes, between about 4 minutes to about 8 minutes, and between about 8 minutes and about 16 minutes. In some embodiments, almost all liquid in the collected filtrate may be evaporated 658B to produce a sediment. The resulting sediment may be collected and lyophilized 659 to substantially remove any residual moisture and produce a powder of imaging agents.

In some embodiments of the invention, the step of separating particles by size 406 can comprise centrifuging steps and no steps requiring porous filters. The centrifuging steps can be carried out after the step 653B of collecting a supernatant. In various embodiments, the centrifuging steps are carried out in accordance with Stoke's law for particle separation via centrifugation. By way of explanation, Stoke's law provides the following relation:

$${\vec{v}}_{a} = \frac{2}{9} \cdot \frac{( {ρ}_{p} - {ρ}_{f} )}{μ} \cdot {d}^{2} \cdot \vec{a} \qquad ( EQ . 1 )$$

where \vec{v}ₐ is the velocity of the particles in motion or settling along an acceleration vector \vec{a}, which can be due to gravity or centrifugation. The density of the particles is denoted as ρₚ, the density of the fluid is denoted as ρ_{f}, the viscosity of the fluid is denoted as μ, and the diameter of the particles is represented by d. For a centrifuge wherein the velocity and acceleration vectors are substantially along a radial line, and wherein centripetal force is responsible for the primary component of acceleration of particles suspended in the fluid, EQ. 1 can be rewritten as a first order differential equation.

$$\frac{d R}{d t} = {C}_{o} {ω}^{2} R \qquad ( EQ . 2 )$$

where R represents the radial displacement of particles from the axis of the centrifuge, ω represents the angular velocity of the centrifuge, and Cₒ collects the constant terms.

$${C}_{o} = \frac{2}{9} \cdot \frac{( {ρ}_{p} - {ρ}_{f} )}{μ} \cdot {d}^{2} \qquad ( EQ . 3 )$$

Equation 2 can be solved by integration. Using EQ. 3 and rearranging terms yields

$${d}_{co} = \sqrt{ln ( \frac{{R}_{f}}{{R}_{o}} ) \frac{9 μ}{2 ( {ρ}_{p} - {ρ}_{f} ) {ω}^{2} t}} \qquad ( EQ . 4 )$$

where Rₒ represents an initial position of a particle in the fluid, R_{f} represents a final position of a particle in the fluid, and t represents the duration of centrifugation at angular velocity ω. Equation 4 can be interpreted as follows. A particle of size d_{co} with a density ρₚ will move from Rₒ to R_{f} for a chosen set of centrifugation conditions, ω, t, μ, and ρ_{f}.

With this understanding of centrifugation, centrifugation conditions can be selected to separate particles by size in a deterministic manner. As an example, suppose a collection of particles, e.g., the particles produced by any of the methods set forth above, is suspended substantially homogenously in a fluid having a viscosity μ and density ρ_{f}. Further the collection of particles has a broad and unknown distribution of sizes. A method of separating the particles by size can comprise dispensing an amount of the fluid into a centrifuge vial. The location Rₜ of the top of the fluid in the vial with respect to the centrifuge's axis of rotation can be measured and used for Rₒ (Rₒ=Rₜ) and the location of the bottom of the vial R_{b} with respect to the centrifuge's axis of rotation can be measured and used for R_{f} (R_{f}=R_{b}). Guided by EQ. 4, one can select an angular velocity ω and centrifugation time t such that particles of size d_{co} will travel the distance from the top of the fluid in the vial to the bottom of the vial. d_{co} can then be interpreted as a cut-off particle size. Particles substantially this size or larger will be sequestered in the pellet or sediment from the process of centrifugation, and particles approximately this size or smaller will remain in the supernatant. With this understanding, EQ. 4 can also be expressed as the following inequality

$${d}_{s} < \sqrt{ln ( \frac{{R}_{b}}{{R}_{t}} ) \frac{9 μ}{2 ( {ρ}_{p} - {ρ}_{f} ) {ω}^{2} t}} \qquad ( EQ . 5 )$$

where dₛ indicates the approximate sizes of particles remaining in the supernatant after centrifugation under a selected set of centrifugation conditions or parameters, Rₜ, R_{b}, ω, t, μ, and ρ_{f}.

It will be appreciated that any of the selected set of centrifugation conditions can be varied to obtain a desired result. For example, the angular velocity c and centrifugation time t are most easily selected by choosing a centrifugation speed (RCF or RPM) and centrifugation duration. It is also possible to change the length of a vial, which can alter R_{b}, and fluid fill height within the vial, which can alter Rₜ. In some embodiments, μ and ρ_{f} are alterable by choosing types of fluids in which the particles are suspended, or by selecting fluid temperatures. In various embodiments, particles suspended in a fluid solution can be separated by size using centrifugation and choosing centrifugation parameters in accordance with EQ. 5 so that particles substantially of a selected size dₛ or smaller remain in the supernatant. Additionally, EQ. 5 indicates that particles substantially of size dₛ and larger will be collected in the sediment or pellet formed during centrifugation.

The step of separating particles by size using centrifugation as described above and in accordance with EQ. 4 or EQ. 5 can be repeated in various manners. In one manner, the supernatant is decanted from the pellet and subjected to an additional step of separating particles by size using centrifugation. In another manner, the pellet is resuspended in a solution and subjected to an additional step of separating particles by size using centrifugation.

Sequential steps of separating the particles by size using multiple steps of centrifugation can produce collections of particles with desired particle size distributions. As an example, a first solution of particles can be subjected to a first step of separating the particles by size in accordance with the method described above and EQ. 5 to produce a first supernatant having particles smaller in size than a first value denoted dₛ₁. The first step of centrifugation will also produce a first pellet. The first supernatant can then be subjected to a second step of separating the particles by size in accordance with the method described above and EQ. 5 to produce a second supernatant having particles smaller in size than a second value denoted dₛ₂. The second step of centrifugation will also produce a second pellet. In various embodiments, the second centrifugation speed will be higher than the first centrifugation speed and dₛ₂ will be smaller than dₛ₁. The second pellet or sediment formed during the second step of centrifugation can then be collected. Particle sizes within this sediment can have sizes between about dₛ₂ and about dₛ₁. Accordingly, dₛ₂ and dₛ₁ can characterize a particle size distribution for the yielded collection of particles from the second pellet. The first pellet and/or the second supernatant can be subjected to further centrifugation steps to yield additional collections of particles.

In some embodiments, the step or steps of separating particles by size can be repeated under substantially similar conditions to refine or improve the uniformity of particles collected in a yielded powder. For example, a sediment or pellet can be resuspended in solution and subjected to substantially the same centrifugation steps which originally produced the pellet. Repetition of steps can reduce contaminants, e.g., particle sizes outside a desired range, within a yielded powder of particles.

It will be appreciated that the separation of particles by size, as depicted in FIG. 6C, can be selectively altered to produce one or more collections of particles with desired particle size ranges. For example, centrifuge speeds at steps 654 and/or 658A may be selected to change the particle size range in a produced collection of particles. In certain embodiments, higher centrifuge speeds will precipitate smaller particles from solution than slower speeds. Additionally, filter pore sizes at step 655 may be selected to change the particle size range in a produced collection of particles. In various embodiments, smaller pore sizes remove smaller particles from solution than larger pore sizes.

### IV-B. Cleaning the Particles

In certain embodiments, a resulting powder of imaging agents is subjected to a step of cleaning 660. In various embodiments, an etching and/or cleaning bath may be used to reduce the thickness of, or remove, any surface oxide layer that may have formed on the particles, or to remove any contaminants that may have collected with the powder during machining or post-machining processes. In some embodiments, an etching bath of hydrofluoric acid can be used to alter the amount of surface oxide on silicon particles. In some embodiments a piranha bath, e.g., a combination of sulfuric acid and hydrogen peroxide, can be used to remove most organic and some inorganic contaminants from the particles. In some embodiments, an RCA-1 bath, e.g., a heated mixture of water, hydrogen peroxide and ammonium hydroxide, may be used to remove most organic contaminants from the particles. In yet additional embodiments, a combination of cleaning methods may be carried out on the particles, e.g., a piranha bath followed by an RCA-1 bath, an etching bath of hydrofluoric acid followed by an RCA-1 bath, etc.

In some embodiments, the particles may be annealed at a high temperature. The step of annealing may be carried out prior to the step of cleaning 660 the particles. In certain embodiments, the annealing may reduce certain defects in the particles. In some embodiments, the annealing may form an oxide layer on the surface of the particles. The oxide layer may extend into the particle, as silicon is converted to silicon dioxide. A subsequent etching step, e.g., etching in a bath of hydrofluoric acid, can remove the oxide layer and reduce the size of each particle. In some embodiments, the steps of annealing and etching can be used to alter the size of the particles.

In some embodiments, the step of cleaning 660 the particles may comprise sterilizing the particles for in vivo use. The process of sterilization may include subjecting the particles to antibacterial or antiseptic agents. In some embodiments, the particles may be stored and/or packaged in sterile containers for shipment.

### IV-C. Characteristics of Yielded Powders

In some embodiments, one or more collections of particles or powders are yielded by the inventive methods of making small particles having long T₁ times. In some embodiments, the distribution of sizes within a yielded powder may be on the order of tens of nanometers, or hundreds of nanometers. Each yielded powder may comprise and be characterized by a range of particle sizes. In certain embodiments, each yielded powder may have a particle size range substantially different from other yielded powders. For example, an embodied method may yield four powders having particle size ranges between about 10 nanometers (nm) and about 100 nm, between about 100 nm and about 200 nm, between about 200 nm and about 400 nm, and between about 400 nm and about 800 nm.

In certain embodiments, each yielded powder may be characterized by an average particle size d_{avg} and/or a particle size distribution d_{dis}. In some embodiments, the average particle size for a yielded powder can be any value between about 1 nm and about 200 nm, between about 200 nm and about 1 micron, and yet between about 1 micron and about 200 microns. In some embodiments, the particle size distribution may be tens of nanometers, or in embodiments hundreds of nanometers. In some embodiments, a yielded powder may have an average particle size d_{avg}, e.g., about 50 nm, about 100 nm, about 150 nm, etc., and the particle size distribution d_{dis} may be expressed as a percentage of the average particle size, e.g., about ±5%, about ±10%, about ±15%, about ±20%, about ±25%, about ±30%, about ±40%, about ±50%, about ±60%, and about ±70%. As an additional example, in certain embodiments, three powders may be produced: a first powder with an average particle size of about 50 nm having a particle sizes ranging between about 30 nm and about 70 nm, a second powder with an average particle size of about 150 nm having a particle sizes ranging between about 120 nm and about 180 nm, and a third powder with an average size of about 300 nm having a particle sizes ranging between about 260 nm and about 340 nm. As a further example, a yielded powder may have an average particle size of about 120 nm, and a particle size distribution of about ±40%. For this powder, the majority of particles will have a size between about 70 nm and about 170 nm.

In some embodiments, a characteristic T₁ time may be associated with a yielded powder in addition to the particle size distribution. In some embodiments, a particular particle size distribution may be offered in plural batches, each batch having a different characteristic T₁ time. In certain aspects, there can be a correspondence or correlation between particle size distribution and characteristic T₁ time. As an example, particle size distributions with a smaller average particle size can have a shorter T₁ time compared to particle size distributions with a larger average particle size. In certain embodiments, the step 406 of separating particles by size can further include a step of determining or measuring a T₁ time associated with a separated particle size distribution.

In some embodiments, the step 406 of separating particles by size can further include measuring and/or verifying a yielded particle size distribution. Either of two methods for measuring and/or verifying the particle size distribution can be employed for this purpose. One method employs dynamic light scattering, while another method utilizes scanning electron microscopy. In certain embodiments, dynamic light scattering (DLS) measurements can be made with commercial apparatus. (Available from Microtrac, Inc., Montgomeryville, Pa.) Such measurements can provide an estimate of particle size distributions within a solution containing a suspension of particles.

In certain embodiments, an estimate of particle size distribution can be obtained by making scanning electron microscope (SEM) measurements of a prepared sample. In some embodiments, a method measuring and/or verifying a particle size distribution of a yield powder of imaging agents comprises preparing a sample for inspection by SEM. To prepare the sample, a dilute suspension of the particles is agitated, e.g., subjected to sonification, to disperse the particles substantially homogeneously in the dilution. An amount of the dilute suspension can then be pipetted onto a vitreous carbon planchett. After evaporation of any solvent, the planchett can be mounted for SEM inspection. Images of the particles disposed on the carbon planchett can be recorded with the SEM, and image analysis software used to determine diameters of particles within an imaged area. A large number of particles, e.g., more than 200, 500 or 1000, can be imaged and analyzed from one or more locations on the planchett to develop statistics about the particles. The number of particles having a measured size value within a size range or bin can be plotted as a function of particle size. For example, a histogram of particle sizes can be produced to determine particle size distribution characteristics, e.g., mean particle size, range of sizes in the distribution, variance of the distribution, etc., for a yielded powder.

It will be appreciated by one skilled in the art that various processing parameters may be altered to change the average particle size, surface properties, and spin-lattice relaxation time T₁ of the yielded particles. The alterable parameters can include, but not be limited to, purity of the starting material, concentration of dopants, machining time, machining speed, machining solvent, gaseous environment in which machining is carried out, ball-mill ball size, sonication power, sonication time, concentration of particles in solution, filtration solvent, centrifugation times, centrifugation speeds, filtration pore size, choice of cleaning and etching baths, post-process annealing, and post-fabrication surface treatments. Any one or combination of these parameters may be selectively altered to obtain particular desired characteristics of the produced particles.

### IV-D. Post-Fabrication Processing and Use

The particles may be subjected to post-fabrication processing after the steps described above. In some embodiments, the particles may be coated or have their surface chemistry altered, e.g., silane or micelle encapsulation. Passivating moieties such as polyethylene glycol (PEG) can provide a protective layer enabling the particle to withstand a living system's natural defense against foreign bodies, and thereby increase the circulation time of the particles in living subjects. In various embodiments, a biologically compatible moiety provides a protective layer which substantially encapsulates the particle. In some embodiments, specific ligands can be conjugated to the particle surface such that the ligand, and conjugated particle, bind specifically to desired target cell types, molecules, or molecular expressions representative of healthy or diseased tissue. As an example, the surface of the particle can be functionalized with a chemical ligand which targets and binds to a particular receptor of interest. The particle may then bind to the receptor when provided into a system containing the target receptors. Some of the coatings or surface treatments may be biodegradable, e.g., provide protection for or activation of the particle for a limited time duration. The coatings or surface treatments may be applied by spray, chemical bath, or evaporation techniques. Examples of methods and techniques for applying coatings to the particles can be found in the published articles by, Kumar P V, Agashe H, Dutta T, Jain N K, “PEGylated dendritic architecture for development of a prolonged drug delivery system for an antitubercular drug,” Curr Drug Deliv, (2007) January; 4(1):11-19; Gref R, Minamitake Y, Peracchia M T, Trubetskoy V, Torchilin V, Langer, R, “Biodegradable long-circulating polymeric nanospheres,” Science, (1994) March 18; 263(5153):1600-1603; Li, H., F. Cheng, et al., “Functionalization of single-walled carbon nanotubes with well-defined polystyrene by ‘click’ coupling,” J Am Chem Soc (2005) 127(41): 14518-14524; and Montet, X., M. Funovics, et al., “Multivalent effects of RGD peptides obtained by nanoparticle display,” J Med Chem (2006) 49(20): 6087-6093, each of which is incorporated by reference in its entirety.

In certain embodiments, untreated and uncoated particles may be used as imaging agents. Uncoated particles processed by the present methods can be delivered into a living system to track, for example, hemodynamic processes, digestive function, and liver biodistributions. Moreover, such uncoated particles can also be loaded into cells, including stem cells, to track the history of the cell in a biological system. Uncoated and untreated particles may biodegrade in the subject over an extended period of time, substantially eliminating the potential for long-term side effects.

In certain embodiments, porous material, e.g., porous silicon, is used as the substantially pure material 520 to form porous particles. In various embodiments, after the porous particles are produced, one or more pharmaceutical drugs may be loaded into the vacancies of the particles. For example, drugs may be absorbed into the pores from a chemical bath. In some embodiments, a method of loading imaging agent particles with drugs can include the steps of receiving porous particles having spin-lattice relaxation times T₁ greater than about 5 minutes, and subjecting the porous particles to a drug-loading process. The drug-loading process can comprise placing the porous particles in a solution containing the desired drug, in which the porous particles absorb an amount of the drug.

In some embodiments, the particles loaded with drugs are delivered to one or more cells, an organism, a specimen, a system, an in vitro assay, or a living subject. In various embodiments, the drug-laden particles can be tracked in vivo. Examples of methods and techniques for loading drugs into the particles can be found in articles by Akerman, M. E., W. C. Chan, et al., “Nanocrystal targeting in vivo,” Proc Natl Acad Sci (2002) USA 99(20): 12617-12621; Simberg, D., T. Duza, et al., “Biomimetic amplification of nanoparticle homing to tumors,” Proc Natl Acad Sci (2007) USA 104(3): 932-936; and Arap, W., R. Pasqualini, et al., “Cancer treatment by targeted drug delivery to tumor vasculature in a mouse model,” Science (1998) 279: 377-380, each of which is incorporated by reference in its entirety.

In some embodiments, particles having long-T₁ times can be incorporated into pharmaceutical tablets, and/or may be chemically conjugated to active ingredients within the tablets. Particles incorporated into pharmaceutical tablets or particles chemically conjugated to pharmaceutical agents can provide diagnostic information about drug delivery and drug history in vivo, and aid in the development of new medications.

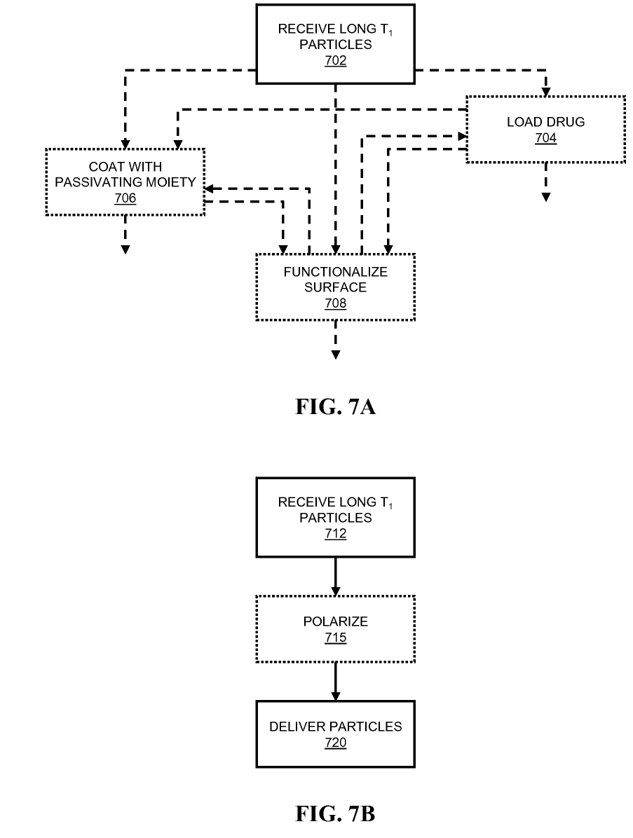

FIG. 7A depicts various post-fabrication methods for particles having long spin-lattice relaxation times. In various embodiments, each of the embodied methods includes the step of receiving particles 702 having spin-lattice relaxation times T₁ greater than about 5 minutes. In certain embodiments, the received particles have relaxation times longer than about 15 minutes, longer than about 30 minutes, longer than about one hour, longer than about two hours, and yet longer than about three hours. In various embodiments, the particles are formed from a substantially pure material comprising at least one chemical element having a T₁ greater than about 5 minutes present in the material. As an example, the particles may be formed from substantially pure silicon having a concentration of the isotope ²⁹Si present in the material.

Any of several steps shown in FIG. 7A may follow the step of receiving particles 702, and in some embodiments, a combination of subsequent steps may be carried out. In some embodiments, a subsequent step may comprise coating the particle with a passivating and/or biologically compatible moiety 706. In some embodiments, a subsequent step may comprise loading a pharmaceutical agent or drug 704, e.g., loading a drug into a porous particle, or conjugating a drug to the surface of a particle. In some embodiments, a subsequent step can comprise chemically functionalizing the surface 708 of the particle. In yet additional embodiments, combinations of the steps can be carried out as depicted in FIG. 7A, for which the dashed lines indicate optional flow paths, and dashed boxes indicate option process steps. For example, in some embodiments, the step of receiving 702 may be followed by the step of functionalizing the surface 708 of the particles, which in turn may be followed by the step of loading a drug 704.

In certain embodiments, the step of chemically functionalizing the surface of the particles 708 may be adapted to treat the exterior surface, for which its properties may be determined substantially by a preceding processing step. For example, if the prior process step was receiving the particles 702 formed from substantially pure silicon material, then the step of chemically functionalizing 708 would be adapted to treat a surface comprising silicon. If the prior process step was coating the particles with a biologically compatible moiety 706, then the step of chemically functionalizing 708 may be adapted to treat a surface comprising an organic compound, such as polyethylene glycol (PEG).

FIG. 7B depicts an embodiment of a method for administering or delivering imaging agent particles having long spin-lattice relaxation times. In various embodiments, the method comprises the step of receiving particles 712 having spin-lattice relaxation times T₁ greater than about 5 minutes. In certain embodiments, the received particles have relaxation times longer than about 15 minutes, longer than about 30 minutes, longer than about one hour, longer than about two hours, and yet longer than about three hours. In various embodiments, the particles are formed from a substantially pure material comprising at least one chemical constituent having a spin-lattice relaxation time T₁ greater than about 5 minutes. In certain embodiments, the received particles may have been modified by one or more post-fabrication methods as depicted in FIG. 7A. For example, the particles may have any of the following characteristics: loaded with a pharmaceutical agent, coated with a biologically compatible moiety, chemically functionalized surface, and any combination thereof. In some embodiments, the particles are received in a non-polarized state, i.e., a state in which the nuclear magnetic moments are randomly oriented. In certain embodiments, the step of receiving particles 712 further comprises sterilizing the particles for delivery in vivo. In some embodiments, the method of delivering the imaging agent particles further includes the step of polarizing the particles 715. The step of polarizing the particles may comprise placing the particles in a substantially uniform and static magnetic field for a period of time. The method of delivering the imaging agent particles may further include the step of delivering 720 a selected quantity of the nanoparticles to one or more cells, an organism, a specimen, a system, an in vitro assay, or a living subject. The step of delivering the particles 720 can comprise delivering them by any one or combination of the following techniques: injection, infusion, ingestion, implantation, absorption and inhalation.

### EXAMPLES

### Example 1

The following example summarizes an embodiment of a method for making silicon particles having spin-lattice relaxation times T₁ of about fifteen minutes. For this embodiment, the average diameter of a particle within the powder is about 350 nanometers.

High-resistivity (greater than about 30 kΩ-cm) undoped silicon wafers having a purity greater than about 99.9999% were obtained and machined in a ball mill using zirconia milling balls having diameters of about 10 millimeters. Ethanol was added as the machining solvent, and the mill was operated at a rotational speed of about 400 revolutions per minute for a period of about 24 hours. During the milling, the wafers were substantially reduced into a collection of particles having various sizes.

The particles were brought up in a stock solution of ethanol after machining. The particles were sonicated in solution, and the solution was left idle for several days. Some of the solution was then dispensed in a vial and centrifuged lightly, at about 3,500 relative centrifugal force (RCF) for about 15 minutes. Particles larger than about 700 nanometers in size pelleted out of the solution and into a first pellet or sediment during centrifugation. A first supernatant remained above the first pellet.

The first supernatant was extracted, placed in a vial, and centrifuged again, at about 3,500 RCF for about 60 minutes producing a second pellet. Particles smaller than about 150 nm in size remained in solution in a second supernatant, which was then decanted, leaving particles of sizes between about 150 nm and about 700 nm in the second pellet.

The second pellet was collected, resuspended in solution, placed in a vial and centrifuged vigorously at about 12,000 rcf for about 10 minutes. During this centrifugation particles precipitated out of the solution and formed a third pellet at the bottom of the vial. The third supernatant was removed from the vial, and the third pellet was collected and dried by lyophilization.

The dried nanoparticles were subsequently etched for about one minute in a hydrofluoric acid bath. This etching reduced the thickness of any surface oxide layer that may have formed during the fabrication process.

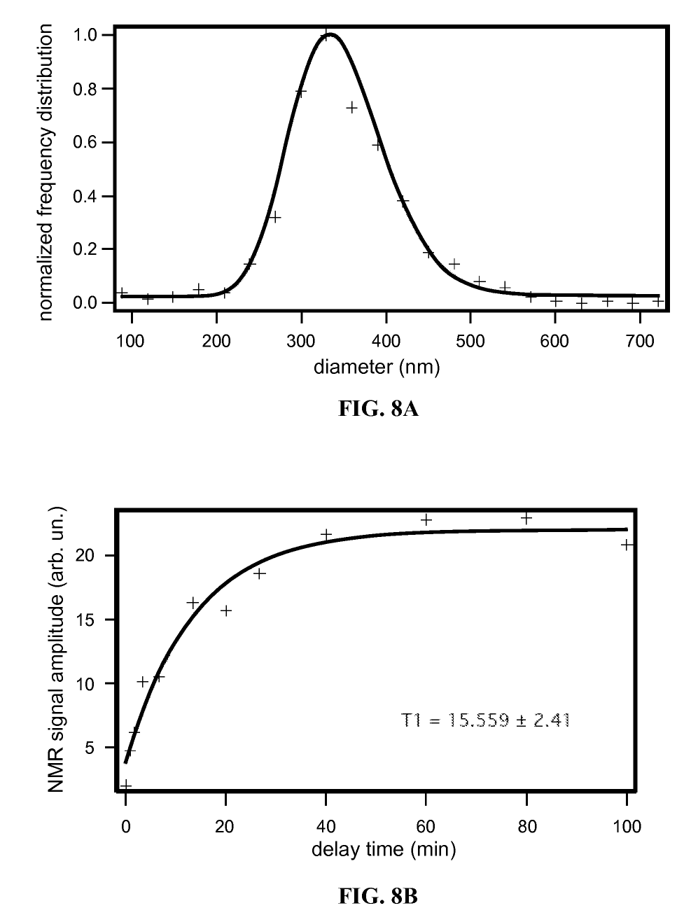

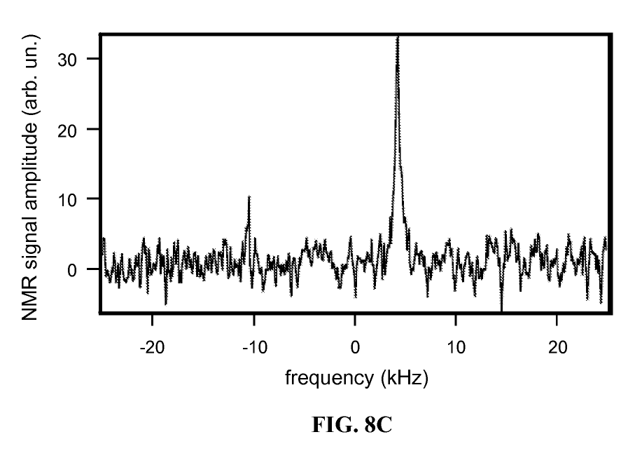

A prepared sample of the yielded nanoparticles was viewed with a scanning-electron microscope (SEM). Multiple images of particles were recorded from random areas of the prepared sample. Image processing software was used to evaluate particle sizes assuming a spherical particle shape, and to record the number and size of particles within the processed image. The resulting data was then processed to yield a particle size distribution for the sample, which is shown in FIG. 8A. The data, shown as crosses, are plotted as a normalized distribution, and a solid line is fit to the data. The data reveals that the average size of the particles within the powder produced by this fabrication method was about 350 nanometers, and that particles within the yielded powder have a range of sizes between about 200 nm and about 550 nm.

Measurements were made of NMR signals produced by the particles as a function of time after initial polarization. The measurements, crosses, are plotted in FIG. 8B and an exponential curve is fit to the data. The results show that the particles produced by the method set forth in this example have a spin-lattice relaxation time T₁ value of about 15 minutes. An example of a measured NMR signal is shown in FIG. 8C.

It will be appreciated that the signal quality available from the silicon nanoparticles having T₁ times greater than about 15 minutes can be significantly better than conventional NMR signals derived from protons (H⁺) in living systems. Since silicon is naturally present in low abundance in living systems, the background noise contributed by native silicon is expected to be low. In various embodiments, silicon particles having long-T₁ times provide signal-to-noise ratios greater than those achieved with conventional proton imagine. In various embodiments, the signal-to-noise ratio provided by the silicon particles is greater than about 1, greater than about 2, greater than about 5, greater than about 10, greater than about 20, greater than about 50, and yet greater than about 100.

### Example 2

An experiment was carried out to demonstrate separation of particles by size using multiple centrifugation steps. For this example, two samples of particles were prepared as described in Example 1 on different dates. However, after light centrifugation at about 3,500 RCF for about 15 minutes, each supernatant was extracted for further study.

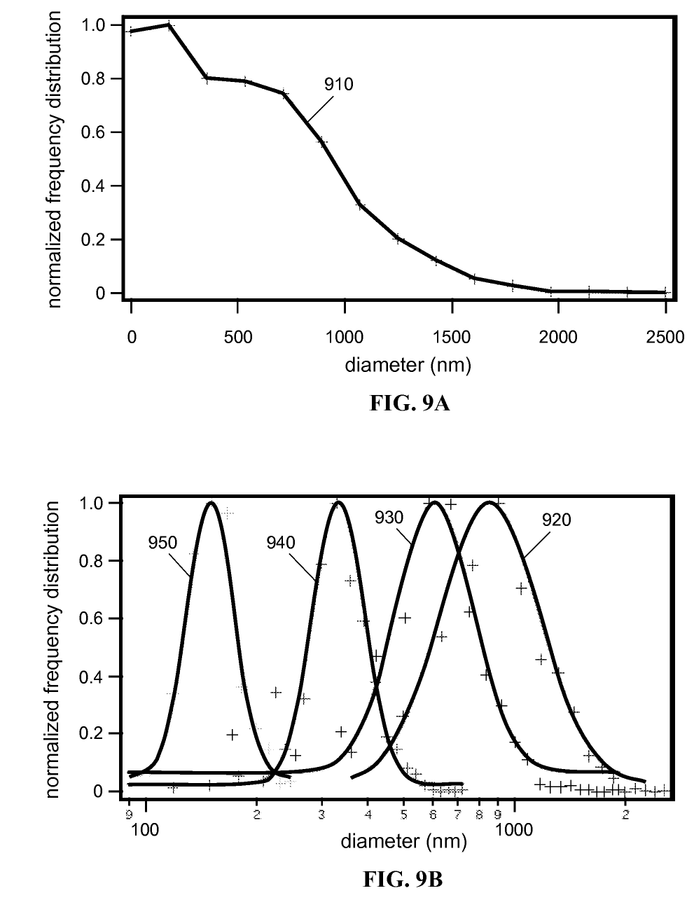

Scanning electron microscope size analysis was carried out for each supernatant, and results are plotted in FIG. 9A for one of the extracted supernatants. Results for the second extracted supernatant were similar. To evaluate the distribution of particle sizes in the supernatant, samples prepared from the supernatant were subjected to scanning electron microscope (SEM) particle size analysis as described in Example 1. Curve 910 represents the measured and normalized particle size distribution of particles within the supernatant after light centrifugation at about 3,500 RCF for about 15 minutes for particles prepared in accordance with the method set forth in Example 1. The average particle size for the collection of particles was found to be about 550 nm.

The supernatant was then subjected to further steps of centrifugation to further separate the particles by size. Typically, the supernatant was subjected to centrifugation at a first speed to produce a first pellet and a first supernatant, and subsequent steps either subjected the first supernatant and/or successively produced supernatants to higher centrifugation speeds or subjected the first pellet and/or successively produced pellets resuspended in solution, to lower centrifugation speeds to further separate particles by size in accordance with EQ. 5. Collections of particles produced according to these methods were subjected to additional SEM particle size analyses. Additional particle-size distribution curves 920, 930, 940, and 950 were recorded and are shown in FIG. 9B.

In certain embodiments, multiple steps of centrifugation yields particles having a size distribution between about 100 nm and about 250 nm, curve 950. In certain embodiments, multiple steps of centrifugation yields particles having a size distribution between about 200 nm and about 600 nm, curve 940. In certain embodiments, multiple steps of centrifugation yields particles having a size distribution between about 350 nm and about 1300 nm, curve 930. In certain embodiments, multiple steps of centrifugation yields particles having a size distribution between about 500 nm and about 2000 nm, curve 920. Additional data for each curve in FIGS. 9A-9B are reported in Table 1.

It will be appreciated from the data of FIGS. 9A-9B and Table 1 that one or more separation techniques can be used to produce a collection of particles with a desired range of sizes. In some embodiments, a method of separating particles by size can comprise one or more steps of filtration using porous filters through which a solution of particles is passed and one or more steps of centrifugation.

TABLE 1

| data curve | D (10%) | D (50%) | D (90%) |
|---|---|---|---|
| 910 | 150 nm | 550 nm | 1180 nm |
| 920 | 460 nm | 950 nm | 1450 nm |
| 930 | 390 nm | 670 nm | 960 nm |
| 940 | 290 nm | 350 nm | 450 nm |
| 950 | 120 nm | 160 nm | 200 nm |

### Example 3

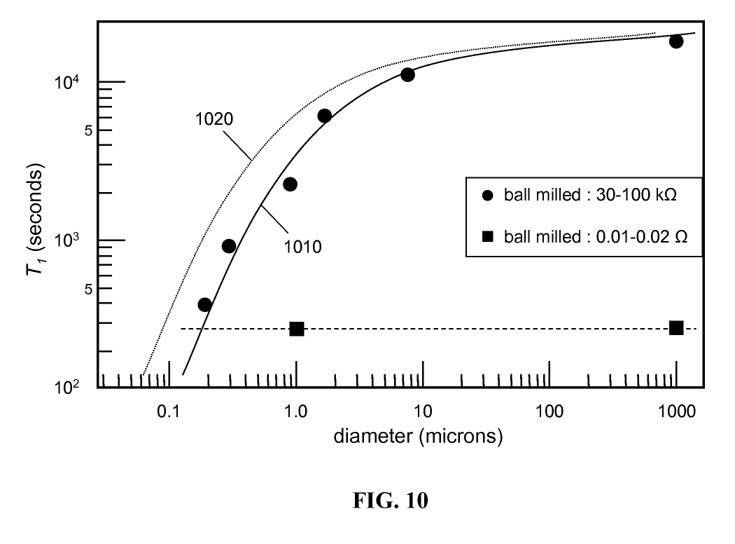

An example was carried out to assess both particle size and T₁ times of collections of particles produced according to the inventive methods. For this example, two types of materials were obtained for processing according to the inventive methods. In one experiment, a low resistivity (between about 0.011-cm and about 0.02 Ω-cm) silicon substrate was obtained and reduced into particles. In a second experiment, a high resistivity (between about 30 kΩ-cm and about 100 kΩ-cm) silicon substrate was obtained and reduced into particles. The particles produced in each experiment were then separated by size according to the inventive methods set forth above. Average particle size was determined for each particle size distribution, and T₁ times were also measured for each particle size distribution. The results obtained for this example are shown in FIG. 10.

For the case of high-resistivity silicon, circles, the data of FIG. 10 indicates that larger particles produced by the inventive methods exhibit longer T₁ times. It is postulated that the step of reducing bulk material to micro- and nanoparticles by ball milling introduces more defects into the crystalline silicon for smaller particles than for larger particles, and that these defects adversely affect the spin-lattice relaxation time. For particle sizes between about 10 microns and about 1 millimeter, the process of making the particles according to the inventive methods set forth above does not substantially affect the material's T₁ time. For particle sizes between about 10 microns and about 1 millimeter T₁ times of about 3 hours were measured.

For particle sizes between about 100 nanometers and about 10 microns, the measured T₁ time varied approximately according to the curve 1010 shown in the graph of FIG. 10. These results suggest that there is a correspondence between particle size and T₁ time. In some embodiments, results such as those shown in FIG. 10 can be used to select collections of particles having a particular T₁ time. For example, if a particular T₁ time is desired, e.g., about 1000 seconds, then a collection of particles with an average size of about 350 nanometers can be selected to provide the desired T₁ time. In some embodiments, a measurement of a T₁ time for a collection of particles of unknown size produced by the inventive methods can be used, in conjunction with data such as that of FIG. 10, to determine an approximate average particle size of the collection of particles.

It will be appreciated that alterations to the inventive methods for making small particles having long T₁ times can alter the results of FIG. 10. For example, an alteration to the milling process or material used may produce particles with T₁ times which fall approximately on a similar, but displaced curve 1020. A new curve can be used to characterize collections particles of unknown T₁ times or unknown average particle size for a particular process.

The results of FIG. 10 also indicate that low resistivity silicon, squares, have short T₁ times, less than about 5 minutes, which are not substantially altered according to particle size. For purposes of comparison, commercially available particles produced by chemical synthesis techniques and known under trade names as Meliorum, NanoAmor, and MTI have T₁ times of at best about 700 seconds.

All literature and similar material cited in this application, including, but not limited to, patents, patent applications, articles, books, treatises, and web pages, regardless of the format of such literature and similar materials, are expressly incorporated by reference in their entirety. In the event that one or more of the incorporated literature and similar materials differs from or contradicts this application, including but not limited to defined terms, term usage, described techniques, or the like, this application controls.

The section headings used herein are for organizational purposes only and are not to be construed as limiting the subject matter described in any way.

While the present teachings have been described in conjunction with various embodiments and examples, it is not intended that the present teachings be limited to such embodiments or examples. On the contrary, the present teachings encompass various alternatives, modifications, and equivalents, as will be appreciated by those of skill in the art.

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process. Furthermore, it is to be understood that the invention encompasses all variations, combinations, and permutations in which one or more limitations, elements, clauses, descriptive terms, etc., from one or more of the listed claims is introduced into another claim. For example, any claim that is dependent on another claim can be modified to include one or more limitations found in any other claim that is dependent on the same base claim. Furthermore, where the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise.

Where elements are presented as lists, e.g., in Markush group format, it is to be understood that each subgroup of the elements is also disclosed, and any element(s) can be removed from the group. It should it be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not been specifically set forth in haec verba herein. It is also noted that the term “comprising” is intended to be open and permits the inclusion of additional elements or steps.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention (e.g., any cell type; any neuronal cell system; any reporter of synaptic vesicle cycling; any electrical stimulation system; any imaging system; any synaptic vesicle cycling assay; any synaptic vesicle cycle modulator; any working memory modulator; any disorder associated with working memory; any method of use; etc.) can be excluded from any one or more claims, for any reason, whether or not related to the existence of prior art.

The claims should not be read as limited to the described order or elements unless stated to that effect. It should be understood that various changes in form and detail may be made by one of ordinary skill in the art without departing from the spirit and scope of the appended claims. All embodiments that come within the spirit and scope of the following claims and equivalents thereto are claimed.

What is claimed is:

1. A method of making particles, the method comprising:
obtaining a substantially pure material comprising at least one constituent having a spin-lattice relaxation time T₁ greater than about 5 minutes;
reducing the substantially pure material into particles in the presence of one or more solvents; and
separating the particles by size to yield one or more collections of particles exhibiting a spin-lattice relaxation time T₁ greater than about 5 minutes.

2. The method as claimed in claim 1, wherein the spin-lattice relaxation time T₁ of a yielded collection of particles is greater than about 15 minutes.

3. The method as claimed in claim 1, wherein the substantially pure material is a material selected from the group consisting of: silicon, silica, silicon carbide, silicon nitride, and carbon.

4. The method as claimed in claim 1, wherein the at least one constituent comprises an isotope selected from the group consisting of: ¹³C, ²⁹Si, and a combination thereof.

5. The method as claimed in claim 1, wherein the stoichiometric purity of the substantially pure material is greater than about 90%.

6. The method as claimed in claim 1, wherein the concentration of the at least one constituent is between about 0.1% and about 100%.

7. The method as claimed in claim 1, wherein the step of reducing comprises reducing bulk material in a machine selected from the following group: a ball mill, a jet mill, a grinding machine, a cutting machine, and any combination thereof.

8. The method as claimed in claim 1, wherein the step of reducing comprises reducing bulk material in a ball mill operated at a speed between about 50 revolutions per minute and about 400 revolutions per minute.

9. The method as claimed in claim 8, wherein the ball mill is operated for a period of time between about 12 hours and about 48 hours.

10. The method as claimed in claim 8, wherein one or more zirconia milling balls having diameters between about 2 mm and about 15 mm are used in the ball mill.

11. The method as claimed in claim 1, wherein the solvent is selected from the group consisting of: water, de-ionized water, distilled water, purified water, ethanol, isopropanol, methanol, and any combination thereof.

12. The method as claimed in claim 1, wherein a yielded collection of particles has an average particle size between about 1 nm and about 200 nm.

13. The method as claimed in claim 1, wherein a yielded collection of particles has an average particle size between about 200 nm and about 1 μm.

14. The method as claimed in claim 1, wherein a yielded collection of particles has an average particle size between about 1 μm and about 200 μm.

15. The method as claimed in claim 1, wherein more than about 90% of the particles within a yielded collection of particles have a size between about 200 nm and about 500 nm.

16. The method as claimed in claim 1 further comprising:
removing contaminants from the surface of the particles.

17. The method as claimed in claim 1 further comprising:
sterilizing the particles.

18. The method as claimed in claim 1, the step of separating the particles by size comprising:
gathering the particles in a solution;
centrifuging the solution to produce a first pellet and a first supernatant; and
subjecting the first supernatant and/or the first pellet to one or more subsequent steps of centrifugation.

19. The method as claimed in claim 18, wherein the maximum particle size dₛ in any of the produced supernatants is selected by choosing centrifugation parameters in accordance with the relation
$${d}_{s} < \sqrt{ln ( \frac{{R}_{b}}{{R}_{t}} ) \frac{9 μ}{2 ( {ρ}_{p} - {ρ}_{f} ) {ω}^{2} t}} wherein$$
Rₜ is substantially the location with respect to the centrifuge's axis of rotation of the top of the fluid containing the particles in a centrifugation vial;
R_{b} is substantially the location with respect to the centrifuge's axis of rotation of the bottom of the vial;
ω is substantially the angular velocity at which the centrifuge is operated;
t is substantially the duration of centrifugation;
μ is substantially the viscosity of the fluid containing the particles;
ρ_{f} is substantially the density of the fluid containing the particles; and
ρₚ is substantially the density of the particles.

20. The method as claimed in claim 1, the step of separating the particles by size comprising:
gathering the particles in a solution;
sonicating the solution;
centrifuging the solution;
decanting a supernatant from the centrifuged solution; and
removing excess liquid from the supernatant.

21. The method as claimed in claim 20 further comprising:
letting the sonicated solution stand without substantial motion for a period between about 12 hours and about 48 hours.

22. The method as claimed in claim 20, wherein the step of centrifuging is carried out at a value between about 2,500 relative centrifugal force and about 4,500 relative centrifugal force, and for a time between about 1 minute and about 90 minutes.

23. The method as claimed in claim 1, wherein the substantially pure material is in a material form selected from the group consisting of: amorphous, crystalline, porous, polycrystalline, nanocrystalline, or co-crystalline.

24. The method as claimed in claim 1, the step of separating the particles by size comprising:
gathering the particles in a solution;
sonicating the solution;
letting the sonicated solution stand without substantial motion for a period of time;
centrifuging the solution;
decanting a first supernatant from the centrifuged solution;
centrifuging the first supernatant to produce a second supernatant and pellet;
decanting the second supernatant; and
removing excess liquid from the pellet to yield a collection of particles.

25. The method as claimed in claim 1, the step of separating the particles by size comprising:
gathering the particles in a solution;
sonicating the solution;
letting the sonicated solution stand without substantial motion for a period of time;
centrifuging the solution;
decanting a first supernatant from the centrifuged solution;
filtering the first supernatant to produce a filtrate;
centrifuging the filtrate to produce a pellet of particles; and
removing excess liquid from the pellet to yield a collection of particles.

26. The method as claimed in claim 25, wherein the filtering is carried out sequentially with filters of gradually reducing pore size.

27. The method as claimed in claim 25, wherein the removing of excess liquid is done by lyophilization.

28. The method as claimed in claim 25, wherein the step of removing of contaminants from the surface of the particles comprises a process step selected from the group consisting of: immersion in hydrofluoric acid, immersion in a mixture of sulfuric acid and hydrogen peroxide, and immersion in a heated mixture of water, hydrogen peroxide and ammonium hydroxide.

29. The method of claim 1, further comprising:
coating the particles with a passivating moiety, the passivating moiety providing a protective layer enabling the particle to withstand a living system's natural defense against foreign bodies.

30. The method of claim 1, further comprising:
chemically functionalizing the surface of the particles with a ligand so that the particle binds specifically to a desired target cell type, molecule, or molecular expression.

31. The method of claim 1, wherein the particles within the yielded collection of particles are porous, and further comprising:
subjecting the porous particles to a drug-loading process, wherein the particles are exposed to a drug to be loaded into the vacancies of the particles.

32. A collection of particles produced by the method of claim 1, the collection of particles having an average particle size between about 1 nm and about 200 μm and a characteristic spin-lattice relaxation time T₁ greater than about 15 minutes.

33. A collection of particles having an average particle size between about 1 nm and about 200 μm, the collection of particles having a characteristic spin-lattice relaxation time T₁ greater than about 15 minutes.

34. The collection of particles as claimed in claim 33, wherein the collection was produced by a method comprising multiple steps of centrifugation.

35. The collection of particles as claimed in claim 33, wherein more than about 90% of the particles have a size within a range between about ±60% of the average particle size.

36. The collection of particles as claimed in claim 33, wherein more than about 90% of the particles have a size within a range between about ±40% of the average particle size.

37. A method of delivering particles to a specimen or subject, the method comprising:
using the collection of particles of claim 33; and
delivering a selected quantity of the particles internally to the specimen or subject.
